(19) United States
(12) Patent Application Publication
(10) Pub. No.: US 2013/0210155 A1
(43) Pub. Date: Aug. 15, 2013

(54) Methods For Selection Of A Naphthenate Solids Inhibitor And Test Kit, And Method For Precipitating Naphthenate Solids

(71) Applicants: Chandrashekhar Khandekar, Katy, TX (US); Suguna Gopal, Dubai (AE); James Smith, Bentley (AU)

(72) Inventors: Chandrashekhar Khandekar, Katy, TX (US); Suguna Gopal, Dubai (AE); James Smith, Bentley (AU)

(21) Appl. No.: 13/496,416

(22) PCT Filed: Sep. 17, 2010
(86) PCT No.: PCT/AU2010/001219

§ 371 Date: Nov. 21, 2012

(30) Foreign Application Priority Data
Sep. 17, 2009 (AU) 2009904522

(51) Int. Cl. G01N 33/28

(52) U.S. Cl. CPC G01N 33/28
USPC 436/60; 422/430

(57) ABSTRACT

The present invention relates to a method for identifying an inhibitor to the formation of naphthenate solids in a liquid hydrocarbon including contacting a sample of the liquid hydrocarbon with an inhibitor and a buffered aqueous solution, observing the extent of formation of naphthenate solids, if any, the extent of formation of naphthenate solids being indicative of the effectiveness of the inhibitor, and repeating the steps, if necessary, until a suitable inhibitor is identified. The present invention also relates to a method for identifying an inhibitor to the formation of naphthenate scale in a liquid hydrocarbon system as well as test kits for use in the methods.

### FIELD OF THE INVENTION

The present invention relates to methods for identifying an inhibitor to the formation of naphthenate solids, particularly calcium naphthenate scale, in a liquid hydrocarbon, for example in oil processing equipment, and test kits for use in such methods. The invention also provides a method for precipitating naphthenate solids, particularly calcium naphthenate scale, from a liquid hydrocarbon, generally irrespective of the source.

### BACKGROUND TO THE INVENTION

The formation of naphthenate solids in crude oil during extraction and refinement presents a plethora of problems. For example, the formation of solids in pipelines may result in the slowing or complete cessation of oil flow. Other problems include:
plugging of chokes, valves, pumps and vessel internals;
the blocking of water legs in separators;
unplanned shutdowns due to hardened deposits causing blockages;
negative impact on water quality due to an increased oil content in the separated water; and
negative impact on injection/disposal well performance.
   Removal of these solids is often difficult, expensive and potentially hazardous to human health.

The formation of solids during crude oil extraction and processing generally results from the reaction of metal cations with indigenous naphthenic acid. In this context, the term “naphthenic acid” is generally considered to refer to complex mixtures of alkyl-substituted acyclic and cyclic carboxylic acids that are generated from in-reservoir biodegradation of petroleum hydrocarbons. They are normal constituents of nearly all crude oils and are typically present in amounts of up to 4% by weight.

The metal cations involved include alkali and alkali-earth metals such as sodium, potassium, calcium and magnesium. Transition metals such as iron may also be involved. However, most solids normally contain a predominant amount of calcium naphthenate species that are formed from a naphthenic acid and/or naphthenate anions and calcium cations. They may precipitate as gummy to hard, solid scale deposits that render control systems inoperable and are detrimental to discharge water and export oil quality. Alternatively, when the acids remain dissolved in the oil they may lower its pecuniary value.

Variations in observed water chemistry, pH, pressure, temperature and shear are generally accepted as the main factors affecting solids formation. As the pressure lowers, more carbon dioxide is lost from the hydrocarbon phase of the crude oil and the pH rises. This increases the degree of dissociation of the naphthenic acids leading to solids precipitation which accumulate at the oil-water interface.

One way to reduce formation of naphthenate solids has been through addition of an acid (either alone or in combination with an inhibitor/demulsifier) during extraction and refinement of crude oil. However, the amount and type of acid, inhibitor, and/or demulsifier needed may vary significantly depending on the source and contents of the crude oil, and the process and refinement conditions utilised.

Various attempts to develop a standardized procedure to evaluate the amount of naphthenic acid inhibitor needed for a particular sample of crude oil or a naphthenate solid deposit have been made. Some have focussed on replicating field conditions and have required significant expenditure on mini-separators and other test equipment to control the variables affecting solids formation (for example, certain pressurized systems permit control and adjustment of a continuous in-situ pH). Existing ‘bottle tests’ while cheaper than mini-separator plant type equipment frequently result in an inability to reproduce solids volume precipitated when used on a particular oil sample or solid deposit under the same test conditions. This may be due to the specific content of the crude oil/deposit or their inability to overcome the natural buffering capacity of naphthenate dissociation. Therefore, there is considerable doubt surrounding the ability of such bottle tests to accurately duplicate the amount of naphthenate solids inhibitor required to prevent precipitation that leads to solid deposit. In addition, their use in identifying the effectiveness of potential naphthenate solid inhibitors is also limited. Thus, it would be desirable to provide a method for readily and reliably forming naphthenate solids from a particular sample of crude oil or naphthenate solid deposit that does not rely on expensive plant type equipment. This may advantageously facilitate the identification of suitable inhibitors for use in the system in question.

### SUMMARY OF THE INVENTION

According to a first aspect of the invention there is provided a method for identifying an inhibitor to the formation of naphthenate solids in a liquid hydrocarbon including:
contacting a sample of the liquid hydrocarbon with an inhibitor and a buffered aqueous solution;
observing the extent of formation of naphthenate solids, if any, the extent of formation of naphthenate solids being indicative of the effectiveness of the inhibitor; and
repeating the steps, if necessary, until a suitable inhibitor is identified.

As already noted, in the context of liquid hydrocarbon bodies, such as crude oil, ‘naphthenic acid’ includes a complex mixture of carboxylic acids. Consequently, the term should be read as such in this specification and should not be construed as particularly limited.

The naphthenate solids may contain any number and type of alkali or alkaline metals as described above. Furthermore, the total solids generated on contacting the sample of liquid hydrocarbon with the buffered aqueous solution may contain other, non-naphthenate type solids, for example precipitated salts such as CaCO₃. However, it is envisaged that the naphthenate solids will predominantly contain calcium naphthenate species. As such, the total solids precipitated, although possibly containing some non-naphthenate type solids, should be indicative of the naphthenic acid present in the original sample.

Importantly, buffering of the aqueous solution guarantees the formation of naphthenate solids at the oil/water interface if the inhibitor is ineffective. Failure to buffer the aqueous solution results in inadequate dissociation of naphthenic acids, an acidic pH shift and reduction (or even elimination) of the amount of naphthenate solids formed.

As used in the present specification, ‘buffered aqueous solution’ refers to a solution whose pH remains essentially unchanged after contact with the sample of the liquid hydrocarbon and the inhibitor. For example, the pH may change by only about 0.05 to 0.6 units, more preferably about 0.1 to 0.5 units and even more preferably from about 0.2 to 0.4 units. Ultimately however, one of ordinary skill in the art will determine when the pH of the solution remains essentially unchanged.

The buffered aqueous solution utilised is not particularly limited. For instance, it may be prepared from naturally occurring water obtained from an oil field or associated processing facility. Alternatively, it may be prepared from an artificial source such as distilled water. If synthetically produced, the buffered aqueous solution generally mimics an ionic species distribution defined by analysis of the naturally occurring formation water. Preferably, the ionic species includes one or more selected from the group consisting of Na⁺, K⁺, Ca²⁺, Mg²⁺, Ba²⁺, Sr²⁺, Cl⁻, SO₄²⁻ and HCO₃⁻. The quantity of each ionic species is not particularly limited. For example, the amount and type of ionic species present may mimic one specific natural aqueous phase associated with the crude oil sample.

In a preferred form, the buffered aqueous solution will have a pH greater than about 6.2 in order to promote formation of naphthenate solids. Although not essential, the buffered aqueous solution will preferably have a pH of between about 6.4 and 8.2, more preferably between 7.0 and 8.2. The pH chosen for the buffered aqueous solution may be somewhat dependent on the particular circumstances, such as the sample to be analysed and/or, the type of inhibitor(s) to be screened.

Any suitable buffering agent may be used. Of course, the agent inevitably chosen will need to provide and maintain the desired pH throughout the method. Preferably, the buffering agent is an organic buffer, even more preferably the buffering agent includes sodium acetate as a conjugate base.

Examples of appropriate buffers are provided in Table 1 below. These are useful in the present invention and include, but are not limited to:

TABLE 1
Recommended acid conjugate base chemistry subject to pH requirements.

| Acid, formula and wt. or volume of standard reagent (mL) | Conjugate base, formula and wt. | pH |
|---|---|---|
| Methyl succinic acid, HO₂CCH₂CH₂CO₂H, 11.8 | Monosodium methylsuccinate, 14 g | 4.1 |
| Acetic acid, CH₃CO₂H, glacial, 5.7 mL | Sodium acetate, CH₃CO₂Na, 8.2 g | 4.75 |
| Potassium hydrogen phthalate, KHC₈H₄O₄, 20.4 g | sodium hydroxide, NaOH, 4.0 g | 4.8 |
| Monosodium methylsuccinate, 15.3 g | Disodium methylsuccinate 17.5 g | 5.6 |
| Monosodium citrate, HOC(CH₂CO₂H)₂CO₂Na, 21.4 g | Disodium citrate, NaOC(CH₂CO₂H)₂CO₂Na, 23.6 g | 5.9 |
| Disodium citrate, HOC(CH₂CO₂Na)₂CO₂H, 23.6 g | Trisodium citrate, HOC(CH₂CO₂Na)₂CO₂Na, 25.8 g | 6.4 |
| Monopotassium phosphate, KH₂PO₄, 12.8 g | Dipotassium phosphate, K₂HPO₄ 15.8 g | 7.2 |
| DL-Cysteine, HSCH₂CH(NH₂)CO₂H, 12.1 g | Sodium DL-Cysteinate, HSCH₂CH(NH₂)CO₂Na, 14.3 g | 8.1 |

The buffered aqueous solution and inhibitor may be contacted with the sample in a number of ways. For example, the contact may involve shearing the buffered aqueous solution, inhibitor and the sample. The rate and duration of shearing is not particularly limited. Preferably, the shear rate is between about 8000 rpm and 10000 rpm and shearing is carried out for a period of about 1 to 10 minutes. Even more preferably, the shear rate of the buffered aqueous solution, inhibitor and sample will be about 9000 rpm for a period of from 1 to 5 minutes. In certain circumstances, shearing may have an adverse impact as an unstable emulsion/precipitate may form. Shearing generally provides adequate results when the pH of the buffered aqueous solution is about 7.0.

An alternative to shearing is to manually shake (for example, by hand shaking) the buffered aqueous solution, inhibitor and the sample. At higher pH values, such as about 8.2, manual shaking is preferred to shearing. The amount of manual shaking required will depend on the nature of both the sample and the buffered aqueous solution. Preferably, the number of shakes will be about 50 to 200 and even more preferably about 100. The buffered aqueous solution and inhibitor may be both sheared and shaken with the sample if desired.

The buffered aqueous solution and inhibitor may also be heated with the sample to assist precipitate formation, if any. If a heating step is included, it is preferred the temperature is between about 50° C. and 80° C., and even more preferably, 65° C. While heating may be performed at any time, preferably the buffered aqueous solution, inhibitor and sample are heated after shearing or hand shaking. Furthermore, the heating duration may be up to 60 minutes and is more preferably about 30 minutes.

It may be desirable to add an acid to the sample under certain circumstances. This may help resolve the interface between the aqueous and oil phases and any precipitate that is formed. The acid may also improve the quality of the water for easier discharge or disposal. Preferably, acetic acid is used.

It may also be desirable to conduct a blank reference test by contacting a sample of the liquid hydrocarbon with the buffered aqueous solution in the absence of the inhibitor and observing the extent of formation of naphthenate solids prior to contacting a sample of the liquid hydrocarbon with an inhibitor.

The sample of liquid hydrocarbon is generally a sample taken from a hydrocarbon body, for example an oil well, at a location within the hydrocarbon body where precipitation of naphthenate solids has substantially not occurred. It will be appreciated, however, that the invention may be applicable to other situations and is therefore not necessarily limited to this embodiment.

According to a second aspect of the invention there is provided a method for identifying an inhibitor to the formation of naphthenate scale in a liquid hydrocarbon system including:
taking a sample of scale from the liquid hydrocarbon system;
solubilising a naphthenate solids component of the scale in an organic solvent to form a naphthenate rich organic solvent;
contacting the naphthenate rich organic solvent with an inhibitor and a buffered aqueous solution;
observing the extent of formation of naphthenate solids, if any, the extent of formation of naphthenate solids being indicative of the effectiveness of the inhibitor; and
repeating the steps, if necessary, until a suitable inhibitor is identified.

The naphthenate component of the scale may be solubilised in an organic solvent by any suitable means. This may involve a number of process steps. In one embodiment naphthenic acid is extracted from the scale with an acid and the extracted naphthenic acid is dissolved in the organic solvent.

Preferably, the method of the second aspect may also include the step of washing the scale with one or more organic solvents prior to acid extraction in order to remove extraneous hydrocarbons from the scale that may interfere with subsequent steps in the method. Suitable organic solvents include those readily miscible with hydrocarbons such as mesitylene, xylene, toluene, heptane and hexane. When xylene is used, the scale is preferably washed with acetone to remove any residual xylene. If desired, the washed scale may be dried at elevated temperature, for example approximately 100° C., prior to acid extraction.

The method of the second aspect may also include assessing the minimum amount of a suitable inhibitor required to inhibit formation of scale by varying the amount of the scale subjected to acid extraction while maintaining a constant concentration of the inhibitor. One will appreciate such assessment may minimise undesirable disposal issues of produced water and result in considerable cost savings.

The concentration of naphthenic acid in the naphthenate rich organic solvent is generally up to about 1%, preferably between about 0.5% and 1%. Preferably, the organic solvent is toluene, although other non-polar organic solvents such as xylene, mesitylene, heptane, hexane and combinations of these may also be used.

The step of contacting the organic solvent with the buffered aqueous solution, for example a buffered produced water, may involve shearing, manual shaking, heating or a combination of one or more of these as described in the first aspect of the invention. Accordingly, these should be read into the second aspect of the invention.

A buffered aqueous solution (or buffered produced water) as described in the first aspect of the invention may be utilised. Whilst the pH may be as low as 6.4, preferably the pH is between about 7.0 to 8.2. This may depend on the particular scale being analysed. When the ionic concentration of naturally occurring water particular to an oil processing and/or refinement location is unknown, a synthetically prepared solution may be utilised instead. As described above in relation to the first aspect of the invention, one of ordinary skill in the art will appreciate that the buffered aqueous solution as used in the second aspect of the invention refers to a solution whose pH remains essentially unchanged after contact with the sample of the naphthenate rich organic solvent and the inhibitor.

Generally, as was the case with the first aspect of the invention, the method may additionally include conducting a blank reference test by contacting a sample of the naphthenate rich organic solvent with the buffered aqueous solution in the absence of the inhibitor and observing the extent of formation of naphthenate solids prior to contacting a sample of the naphthenate rich organic solvent with an inhibitor.

The methods may be used to test any potential inhibitor of naphthenate solids in a liquid hydrocarbon. Broadly speaking, preferred inhibitors are linear or cyclic alkoxylated amines. Examples of this type of inhibitor are alkoxylated fatty amines with a carbon chain length from C₁₀-C₂₄, alkyldiamine ethoxylates, tallowalkylamine ethoxylate propoxylates and quaternary amines of the type:

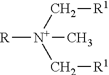

wherein R₁ is (CH₂CH₂O)ₙH and R is a saturated or unsaturated alkyl chain with carbon numbers varying from C₁₀-C₁₆ and having an average number of ethoxylate units of from 10 to 20.

Alternatively, the inhibitor may be a fatty amine with a carbon chain length between C₁₂-C₂₄.

According to a third aspect of the invention there is provided a test kit for use in a method for identifying an inhibitor to the formation of naphthenate solids in a liquid hydrocarbon, the test kit including:
an acid and conjugate base for buffering an aqueous solution containing one or more ionic species selected from the group consisting of Na⁺, K⁺, Ca²⁺, Mg²⁺, Ba²⁺, Sr²⁺, Cl⁻, SO₄²⁻ and HCO₃ to a pH of 6.4 to 8.2;
a plurality of inhibitors preselected based on the nature of the liquid hydrocarbon; and
at least one vessel in which a sample of liquid hydrocarbon may be contacted with an inhibitor and a buffered aqueous solution formed from the acid, conjugate base and aqueous solution.

The buffered aqueous solution may be as described above in respect of the first and second aspects of the invention. In some embodiments this prepared from formation water associated with the liquid hydrocarbon to be tested, or is prepared from a synthetic water that includes ionic species at concentrations representative of the formation water associated with the liquid hydrocarbon to be tested.

As described above, the inhibitors preferably include at least one linear or cyclic alkoxylated amine, for example an alkoxylated fatty amine with a carbon chain length from C₁₀-C₂₄, alkyldiamine ethoxylates, tallowalkylamine ethoxylate propoxylates and/or quaternary amines of the type:

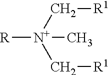

wherein R₁ is (CH₂CH₂O)ₙH and R is a saturated or unsaturated alkyl chain with carbon numbers varying from C₁₀-C₁₆ and having an average number of ethoxylate units of from 10 to 20.

The inhibitors may also include at least one fatty amine with a carbon chain length between C₁₂-C₂₄.

In a certain embodiment, the method is for identifying an inhibitor to the formation of calcium naphthenate scale in a liquid hydrocarbon system, the test kit additionally including:

an acid;

an organic solvent;
at least a first vessel for solubilising a naphthenate component of the scale into the organic solvent to provide a naphthenate rich organic solvent; and
at least a second vessel in which the naphthenate rich organic solvent may be contacted with an inhibitor and the buffered aqueous solution.

According to this embodiment, which is closely related to the method of the second aspect of the invention described above, as will be appreciated by those of skill in the art, the acid is preferably selected from the group consisting of organic acids, such as acetic acid, or inorganic acids, such as hydrochloric acid. The organic solvent is preferably selected from the group consisting of mesitylene, xylene, toluene, heptane and hexane.

As described above in relation to the first and second aspects of the invention, the buffered aqueous solution as used in the third aspect of the invention refers to solution whose pH remains essentially unchanged after contact with the sample of the inhibitor and the liquid hydrocarbon or naphthenate rich organic solvent as the particular instance requires.

Embodiments of the invention will now be discussed in more detail with reference to the following examples which are provided for exemplification only and which should not be considered limiting on the scope of the invention in any way.

### DETAILED DESCRIPTION OF THE DRAWINGS

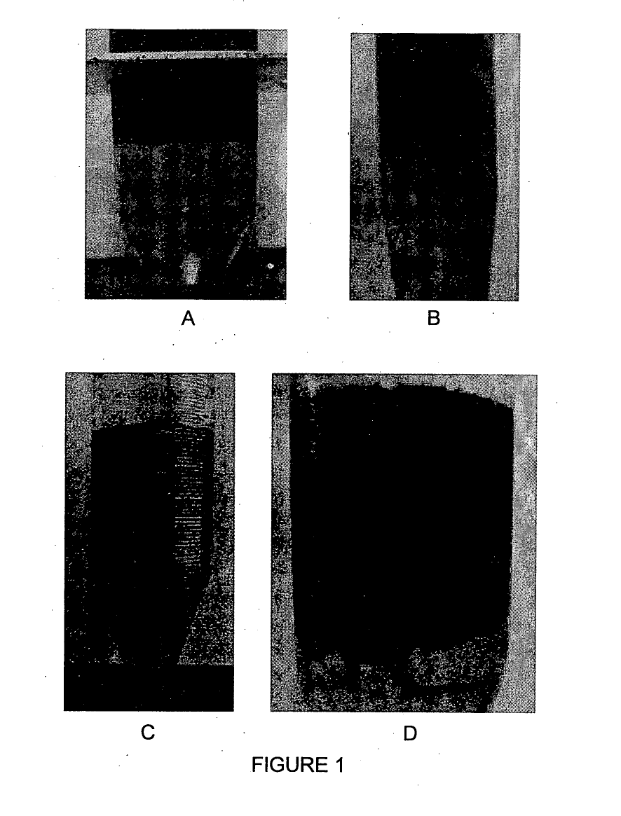

FIG. 1 illustrates unsuccessful (A and B) and successful (C and D) re-formation of naphthenate solids in accordance with a preferred embodiment of the third aspect of the present invention.

### EXAMPLES

### (1) General Test Method for Precipitating a Representative Amount of Calcium Naphthenate from a Liquid Hydrocarbon

Preparation of Buffered Aqueous Solutions (i.e. Produced Water)

Two aqueous samples of (A and B) with differing quantities of ionic species were prepared in accordance with Table 2. Each solution mimicked the total dissolved solids found in produced water samples obtained from two different crude oil samples.

TABLE 2
Composition of Aqueous Solutions A and B.

| | |
|---|---|
| Ionic | Ionic Concentration (mg/l) |

| Species | Aqueous Solution A | Aqueous Solution B |
|---|---|---|
| Na⁺ | 19140 | 12899 |
| K⁺ | 440 | 10291 |
| Ca²⁺ | 1070 | 596 |
| Mg²⁺ | 215 | 165 |
| Ba²⁺ | 250 | — |
| Sr²⁺ | 110 | — |
| Cl⁻ | 30480 | 19505 |
| SO₄²⁻ | 0 | 842 |
| HCO₃⁻ | 500 | 2240 |

Initially, the pH of each aqueous solution in Table 1 was higher than 6.2. The pH was reduced to 5.5 and each aqueous solution was divided into 3 portions. The pH of each portion was then raised to 6.4, 7.0 or 8.2 with conjugate base sodium acetate prior to further use. This is thought to mimic the naturally occurring pH shift whilst maintaining field condition which maintains pH throughout testing. In addition, it is thought that this pH reduction mitigates any non-representative bicarbonate scale formation brought about by the higher pH noted in the synthetic brine which may in turn inhibit any calcium naphthenate co-precipitation.

### Formation and Analysis of Naphthenate Solids

To 50 ml of a crude oil sample taken from a hydrocarbon body was added 50 ml of buffered aqueous solution at a particular pH (6.4, 7.0 or 8.2) and the mixture subjected to a shear rate of 9000 rpm for 5 minutes to produce a stable emulsion. In a separate analysis, the mixture was subjected to 50-200 handshakes. The mixing method which produced the maximum precipitation was analysed further. Following either shearing or hand shaking, the mixture was transferred to a 100 ml centrifuge tube and heated in a water bath at 65° C. for 30 minutes. In all cases, a thick emulsion with differing amounts of colloidal particles was observed at the oil-water interface.

The aqueous and oil phases were separated from the interface. The pH of the buffered aqueous phase was essentially unchanged (see Table 3 below). The interface was transferred onto a pre-weighed filter paper and repeatedly washed with xylene to remove any non-naphthenate emulsion and dried in a forced air oven at 80° C. for 6 hours. The dried interface was visually inspected and the appearance of the residual naphthenate solids noted (Table 3). The dried interface was washed with acetic acid and the washings were collected into a pre-weighed beaker. The washings were evaporated in a forced air oven at 80° C. for 4 hours and the naphthenate solids weighed (Table 3).

TABLE 3
Results from the general test method using aqueous solution B.

| | | | Appearance of the |
|---|---|---|---|
| Run No. | pH initial | pH final | residue |
| 1 | 6.4 | 6.25 | gummy |
| 2 | 7.0 | 6.80 | slightly gummy with |
| | | | mostly solid character |
| 3 | 8.2 | 7.75 | solid |

### Visual Observations and Quantitative Results

pH 6.4:

A heavy emulsion was noted with relatively few colloidal particles compared to when aqueous solutions with higher pH values were used. After washing with xylene and drying, the residue was gummy and did not contain any hard solids. There was no difference in the level of precipitation between the mechanical shearing and the hand shaking.

pH 7.0:

A small amount of emulsion was observed with a relatively significant concentration of colloidal particles. The precipitate was solid in character. Subsequent xylene washing produced a thick gummy residue of solids. After acid washing and drying, the solids remained slightly gummy. There was no difference in the level of solids formation between the mechanical shearing and the hand shaking.

pH 8.2:

Markedly different emulsion and precipitate content was observed compared to when aqueous solutions having a pH of 6.4 or 7.0 were used. Upon mixing cessation, an immediate separation of the oil and aqueous phases and a thick emulsion at the interface were observed. After washing with acid and drying, a 2.5-fold increase in the amount of solid particles was found (relative to the pH 7.0 test). In contrast to the pH 7.0 test, the residue was found to be a solid precipitate with a limited amount of gummy residue.

Without wishing to be bound by any theory, at higher pH values there is an increased deprotonation of naphthenic acids. The tendency to form an emulsion is reduced as solids precipitation increases, and the aqueous phase more rapidly separates. This would be dependent on the amount of shearing and the inclusion of any other natural emulsifiers present within the produced crude.

### (2) Comparative Inhibitor Screening to Identify an Inhibitor to the Formation of Naphthenate Solids in a Liquid Hydrocarbon

The general test method employing a buffered aqueous solution at pH 8.2 (as described in (1) above) was used to screen the effectiveness of a series of potential naphthenate solids inhibitors on two crude oil samples X and Y taken from different hydrocarbon bodies. In some screening runs, the effect of co-adding 200 ppm of acetic acid with the inhibitor was examined. The results are shown in tables 4-7 for oil sample X and tables 8 and 9 for oil sample Y.

TABLE 4
Effect of 1000 ppm of various inhibitors on naphthenate solids formation
in crude oil sample X at a tested pH of 8.2.

| | 1000 ppm inhibitor | | | |
|---|---|---|---|---|
| | | | | final |
| Inhibitor | oil | water | interface | pH |
| Blank | emulsified | oil in water/dirty | baggy with | 7.70 |
| | | appearance | solids | |
| Akzo-1 | clear | opaque | sharp | 7.97 |
| Akzo-2 | emulsified | oil in water/dirty | baggy with | 7.58 |
| | | appearance | solids | |
| Akzo-3 | clear | slightly opaque | sharp | 7.99 |
| Akzo-4 | clear | oil in water | mild emulsion | 6.80 |
| Akzo-5 | not clear | clear | thick emulsion | 7.75 |
| Akzo-6 | clear | oil in water | mild emulsion | 7.95 |
| Rosa-A | clear | clear | sharp | 8.02 |
| Armohib-28 | not clear | dirty water | thick emulsion | — |
| Armohib-31 | not clear | dirty water | thick emulsion | — |
| Ethoduomeen | emulsified | dirty | thick baggy | — |
| T/22 | | | | |
| Ethomeen | bright | oily | baggy | — |
| O/12 LC | | | | |
| Ethoduomeen | emulsified | dirty | thick baggy | — |
| T13 | | | | |
| Ethoduomeen | emulsified | dirty | thick baggy | — |
| OV/13 | | | | |
| Ethomeen OV/22 | emulsified | dirty | thick baggy | — |
| Ethomeen T/20 | emulsified | dirty | thick baggy | — |
| Ethomeen HT/12 | emulsified | oily | thick baggy | — |
| Ethomeen T/12 | thick oil | clear | slightly | — |
| Ethomeen OV/17 | not working | | | — |
| Ethomeen O/12 | baggy | clear | not clear | — |
| Ethomeen T/12E | baggy | clear | not clear | — |
| Ethomeen T/15 | complete emulsion | | | — |
| Ethomeen T/25 | complete emulsion | | | — |
| Ethomeen HT/60 | complete emulsion | | | — |
| Triameen T | complete emulsion | | | — |
| Triameen OV | complete emulsion | | | — |
| Triameen YT | complete emulsion | | | — |
| Tetrameen OV | complete emulsion | | | — |
| Tetrameen T | complete emulsion | | | — |
| Crodafos HCE | bright | dirty | emulsion + | — |
| | | | solids | |
| Crodafos T5A | bright | dirty | emulsion + | — |
| | | | solids | |
| Crodafos N5A | bright | dirty | emulsion + | — |
| | | | solids | |

| | | |
|---|---|---|
| Crodafos N3A | not working | — |
| UK | | |
| ATPHOS 3226 | not working | — |

TABLE 5
Effect of 1000 ppm of various inhibitors and 200 ppm acetic acid on
naphthenate solids formation in crude oil sample X at a tested pH of 8.2.

| | 1000 ppm inhibitor and 200 ppm acetic acid | | | |
|---|---|---|---|---|
| Inhibitor | oil | water | interface | final pH |
| Blank | emulsified | opaque water | baggy | 7.08 |
| Akzo-1 | bright | opaque | sharp | 7.09 |
| Akzo-2 | emulsified | opaque water | baggy | — |
| Akzo-3 | bright | slightly opaque | sharp | 7.04 |
| Akzo-4 | bright | slightly improved | sharp | 6.42 |
| Akzo-5 | hazy | clear | thick emulsion | 7.01 |
| Akzo-6 | bright | slightly improved | sharp | 7.08 |
| Rosa-A | bright | clear | sharp | 7.01 |
| Armohib-28 | hazy | dirty water | thick emulsion | — |
| Armohib-31 | hazy | dirty water | thick emulsion | — |

TABLE 6
Effect of 2000 ppm of various inhibitors on naphthenate solids formation
in crude oil sample X at a tested pH of 8.2.

| | 2000 ppm inhibitor | | | |
|---|---|---|---|---|
| Inhibitor | oil | water | interface | final pH |
| Blank | emulsified | dirty | thick baggy | 7.96 |
| Ethoduomeen T/22 | emulsified | dirty | thick baggy | — |
| Ethomeen O/12 LC | bright | improved | slightly baggy | — |
| Ethoduomeen T13 | emulsified | clear | baggy | — |
| Ethoduomeen OV/13 | emulsified | clear | baggy | — |
| Ethomeen OV/22 | emulsified | dirty | thick baggy | — |
| Ethomeen T/20 | emulsified | dirty | thick baggy | — |
| Ethomeen HT/12 | emulsified | oily | baggy | — |
| Ethomeen T/12 | improved | very clear | slight haze | 8.01 |
| Ethomeen OV/17 | not working | | | — |
| Ethomeen O/12 | — | clear | not clear | 7.75 |
| Ethomeen T/12E | — | clear | not clear | 7.74 |
| Ethomeen T/15 | complete emulsion | | | — |
| Ethomeen T/25 | complete emulsion | | | — |
| Ethomeen HT/60 | complete emulsion | | | — |
| Triameen T | bright | dirty | sharp | — |
| Triameen OV | bright | dirty | sharp | — |
| Triameen YT | bright | dirty | bulky | — |
| Tetrameen OV | bright | dirty | soapy | — |
| Tetrameen T | bright | dirty | sharp | — |
| Crodafos HCE | bright | dirty | emulsion + | — |
| | | | solids | |
| Crodafos T5A | bright | dirty | emulsion + | — |
| | | | solids | |
| Crodafos N5A | bright | dirty | emulsion + | — |
| | | | solids | |
| Crodafos N3A UK | not working | | | — |
| ATPHOS 3226 | bright | dirty | thick | — |

TABLE 7
Effect of 2000 ppm of various inhibitors and 100 ppm acetic acid on
naphthenate solids formation in crude oil sample X at a tested pH of 8.2.

| | 2000 ppm inhibitor and 100 ppm acetic acid | | | |
|---|---|---|---|---|
| Inhibitor | oil | water | interface | final pH |
| Blank | emulsified | dirty | thick baggy | 7.96 |
| Ethoduomeen T/22 | not working | | | — |
| Ethomeen O/12 LC | clear oil/interface and oily water | | | — |
| Ethoduomeen T13 | very thick interface | | | — |
| Ethoduomeen OV/13 | very thick interface | | | — |
| Ethomeen OV/22 | very thick interface | | | — |
| Ethomeen T/20 | very thick interface | | | — |
| Ethomeen HT/12 | bright | bright | sharp | 7.02 |
| Ethomeen T/12 | bright | bright | sharp | 7.02 |
| Ethomeen OV/17 | bright | dirty | sharp | — |

| | | |
|---|---|---|
| Ethomeen O/12 | acid pushed the emulsion | — |
| | into the interface | |
| Ethomeen T/12E | acid pushed the emulsion | — |
| | into the interface | |
| Ethomeen T/15 | complete emulsion | — |
| Ethomeen T/25 | complete emulsion | — |
| Ethomeen HT/60 | complete emulsion | — |
| Triameen OV | no improvement with acid addition | — |
| Tetrameen T | no improvement with acid addition | — |
| Crodafos HCE | very thick interface | — |
| Crodafos T5A | very thick interface | — |
| Crodafos N5A | very thick interface | — |

TABLE 8
Effect of 2000 ppm of various inhibitors on naphthenate solids formation
in crude oil sample Y at a tested pH of 8.2.

| | 2000 ppm inhibitor | | | |
|---|---|---|---|---|
| Inhibitor | oil | water | interface | final pH |
| Blank | dark | dirty | bulky | 7.88 |
| Rosa A/Akzo 6/ | dark | slight oil in water | loose | 7.34 |
| MeOH (1:1:2) | | | | |
| Rosa-A | tight | slight oil in water | loose | 7.8 |
| | emulsion | | | |
| Akzo-1 | complete emulsion | | | — |
| Akzo-2 | complete emulsion | | | — |
| Akzo-3 | complete emulsion | | | — |
| Akzo-4 | tight | slight oil in water | loose | 7.02 |
| | emulsion | | | |
| Akzo-5 | not working | | | — |
| Akzo-6 | tight | clear water | loose | 7.8 |
| | emulsion | | | |
| Ethoduomeen T/22 | complete emulsion | | | — |
| Ethomeen O/12 LC | complete emulsion | | | — |
| Ethoduomeen T13 | complete emulsion | | | — |
| Ethoduomeen | complete emulsion | | | — |
| OV/13 | | | | |
| Ethomeen OV/22 | complete emulsion | | | — |
| Ethomeen T/20 | complete emulsion | | | — |
| Ethomeen HT/12 | dark | slight oil in water | loose | 7.5 |
| Ethomeen T/12 | dark | clear water | better | 7.6 |
| Ethomeen OV/17 | dark | oil in water | solids and | 7.73 |
| | | | emulsion | |
| Ethomeen O/12 | dark | clear water | sharp | 7.62 |
| Ethomeen T/12E | dark | slight oil in water | loose | 7.7 |
| Ethomeen T/15 | dark | clear water | loose | 7.68 |
| Ethomeen T/25 | complete emulsion | | | — |
| Ethomeen HT/60 | dark | slight oil in water | very loose | 7.54 |
| Triameen T | dark | clear water | loose | 7.63 |

| | | |
|---|---|---|
| Triameen OV | complete emulsion | — |
| Triameen YT | complete emulsion | — |
| Tetrameen OV | complete emulsion | — |
| Tetrameen T | complete emulsion | — |
| Crodafos HCE | complete emulsion | — |
| Crodafos T5A | complete emulsion | — |
| Crodafos N5A | complete emulsion | — |
| Crodafos N3A UK | complete emulsion | — |

TABLE 9
Effect of 2000 ppm of various inhibitors and 100 ppm acetic acid on
naphthenate solids formation in crude oil sample Y at a tested pH of 8.2.

| | 2000 ppm inhibitor and 100 ppm acetic acid | | | |
|---|---|---|---|---|
| Inhibitor | oil | water | interface | final pH |
| Blank | dark | dirty | bulky | 7.88 |
| Rosa-A | dark | clear | loose | 7.51 |
| Akzo-1 | complete emulsion | | | — |
| Akzo-2 | complete emulsion | | | — |
| Akzo-3 | complete emulsion | | | — |
| Akzo-4 | dark | clear | loose | 6.81 |
| Akzo-5 | not working | | | — |
| Akzo-6 | dark | clear | loose | 7.21 |
| Ethoduomeen T/22 | complete emulsion | | | — |
| Ethomeen O/12 LC | complete emulsion | | | — |
| Ethoduomeen T13 | complete emulsion | | | — |
| Ethoduomeen OV/13 | complete emulsion | | | — |
| Ethomeen OV/22 | complete emulsion | | | — |
| Ethomeen T/20 | complete emulsion | | | — |
| Ethomeen HT/12 | little improvement | | | 7.22 |
| Ethomeen T/12 | dark | clear | improved | 7.34 |
| Ethomeen OV/17 | little improvement | | | 7.2 |
| Ethomeen O/12 | little improvement | | | 7.31 |
| Ethomeen T/12E | little improvement | | | 7.32 |
| Ethomeen T/15 | little improvement | | | 7.19 |
| Ethomeen T/25 | complete emulsion | | | — |
| Ethomeen HT/60 | oil and solids | | | — |
| Triameen T | dark | improved | loose | 7.2 |

| | | |
|---|---|---|
| Triameen OV | complete emulsion | — |
| Triameen YT | complete emulsion | — |
| Tetrameen OV | complete emulsion | — |
| Tetrameen T | complete emulsion | — |
| Crodafos HCE | complete emulsion | — |
| Crodafos T5A | complete emulsion | — |
| Crodafos N5A | complete emulsion | — |
| Crodafos N3A UK | complete emulsion | — |
| ATPHOS 3226 | complete emulsion | — |

### Effective Solids Inhibitors of Crude Oil Sample X

The Comparative Inhibitor Screening Identified Four Possible Inhibitors:
Akzo-3 at 1000 ppm produced a sharp interface and slightly opaque water. After 200 ppm acetic acid treatment there was a slight improvement in water quality;
Rosa-A at 1000 ppm produced a sharp interface free from solids and emulsion, along with clear water. After 200 ppm acetic acid treatment the pH was above 7 and was thus favourable for discharge;
Ethomeen T/12 at 2000 ppm with 200 ppm acetic acid had a very sharp interface with clear oil and water; and
Ethomeen HT/12 at 2000 ppm with 200 ppm acetic acid also showed acceptable results.

### Effective Solids Inhibitors of Crude Oil Sample Y

The comparative inhibitor screening identified three possible inhibitors:
Akzo-6 at 2000 ppm produced a slightly loose interface with marginal emulsion which disappeared upon addition of 200 ppm acetic acid;
Rosa-A at 2000 ppm also produced a slightly loose interface with marginal emulsion which disappeared by the addition of 200 ppm acetic acid. However, the addition of 200 ppm acetic acid also improved the water quality compared to Akzo-6;
Ethomeen T/12 at 2000 ppm gave very clear water. However, the oil was dark and thick compared to Akzo-6 and Rosa-A. After adding 200 ppm acetic acid the interface improved from loose to being clear and sharp. Advantageously, the addition of acetic acid also had little impact on the pH of the water which remained above 7.

### (3) General Test Method for Identifying an Inhibitor to the Formation of Naphthenate Solids Deposits in Oil Processing Equipment

### Preliminary Purification

A sample of a naphthenate solids deposit was washed repeatedly with xylene to remove unwanted hydrocarbons and other extraneous organic materials. The deposit was then washed with acetone, dried at 100° C. for 12 hours and crushed to a homogenous powder.

### Extraction and Redissolution

To a sample of a naphthenate solids deposit was added either an organic acid, preferably acetic acid, or an inorganic acid, preferably hydrochloric acid, to extract naphthenic acid from the deposit. The mixture was filtered and the insoluble materials (for example, sand and bitumen) washed with 1% organic acid, preferably acetic acid, or inorganic acid, preferably hydrochloric acid, in toluene to ensure complete extraction and filtered again. The combined filtrates were evaporated to dryness and a 0.5% to 1% solution of the naphthenic acid extract in toluene was prepared. The insoluble materials may be dried and weighed to estimate the level of non-naphthenic acid species present in the deposit.

### Preparation of Buffered Aqueous Solution

An aqueous solution that is a sample of produced water or mimics the ionic concentration of produced water particular to an oil processing and/or refinement location is prepared.

In either case, a predetermined amount of a buffering agent (as described above) is added to the aqueous solution. The pH value will depend on the deposit to be analysed and may be determined via computer simulation of the water analysis and facility operating conditions. Usually the pH will be between 7.0 and 8.2.

### Reformation of Naphthenate Solids (Blank Reference)

To 50 ml of the naphthenic acid-containing toluene solution was added 50 ml of the buffered aqueous solution. The mixture was manually shaken approximately 100 times before placing in a water bath between about 50° C. and 80° C. for 30 minutes. Of course, these conditions may require adjustment to optimize the reformation. FIG. 1 illustrates unsuccessful (A and B) and successful (C and D) re-formation of naphthenate solids.

### Screening of Inhibitors

The blank reference procedure was repeated except that a predetermined quantity of an inhibitor was added to the hydrocarbon solution prior to mixing with the buffered aqueous solution. The mixture was observed at 1, 5, 10, 20 and 30 minute intervals. Suitable inhibitors produced a clear water phase and sharp inter phase relative to the blank reference.

One or more inhibitors may be combined in different amounts and/or ratios to optimize the inhibition of naphthenate solids reformation.

If desired, the minimum amount of inhibitor(s) required to inhibit formation of a naphthenate solids deposit may be assessed by varying the amount of the naphthenate solids deposit subjected to acid extraction while maintaining a constant concentration of the inhibitor(s).

It will of course be realised that the above has been given only by way of illustrative example of the invention and that all such modifications and variations thereto as would be apparent to persons skilled in the art are deemed to fall within the broad scope and ambit of the invention as herein set forth.

1. A method for identifying an inhibitor to the formation of naphthenate solids in a liquid hydrocarbon including:
contacting a sample of the liquid hydrocarbon with an inhibitor and a buffered aqueous solution;
observing the extent of formation of naphthenate solids, if any, the extent of formation of naphthenate solids being indicative of the effectiveness of the inhibitor; and
repeating the steps, if necessary, until a suitable inhibitor is identified.

2. The method of claim 1, wherein the pH of the buffered aqueous solution remains essentially unchanged after contact with the sample of the liquid hydrocarbon and the inhibitor.

3. The method of claim 1, wherein the buffered aqueous solution has a pH greater than about 6.2, preferably between about 7.0 and 8.2.

4. The method of any claim 1, wherein the buffered aqueous solution is buffered with an organic conjugate, preferably sodium acetate.

5. The method of claim 1, wherein the buffered aqueous solution, which is preferably produced water, contains one or more ionic species selected from the group consisting of Na₊, K⁺, Ca²⁺, Mg²⁺, Ba²⁺, Sr²⁺, Cl⁻, SO₄²⁻ and HCO₃⁻.

6. The method of claim 1, wherein the step of contacting the sample of liquid hydrocarbon with the inhibitor and buffered aqueous solution includes shearing the sample of liquid hydrocarbon and the inhibitor and buffered aqueous solution at a shear rate of from 8000 rpm to 10000 rpm, preferably for a period of from 1 to 10 minutes at about pH 7.0.

7. The method of claim 1, wherein the step of contacting the sample of liquid hydrocarbon with the inhibitor and buffered aqueous solution includes manually shaking the sample of liquid hydrocarbon and the inhibitor and buffered aqueous solution, preferably for 50 to 200 shakes, more preferably for about 100 shakes at about pH 8.2.

8. The method of claim 1, including the step of heating the sample of liquid hydrocarbon and the inhibitor and buffered aqueous solution at a temperature between about 50° C. and 80° C., preferably for a period of up to 60 minutes, and more preferably for a period of up to 30 minutes.

9. The method of claim 1, including conducting a blank reference test by contacting a sample of the liquid hydrocarbon with the buffered aqueous solution in the absence of the inhibitor and observing the extent of formation of naphthenate solids prior to contacting a sample of the liquid hydrocarbon with an inhibitor.

10. A method for identifying an inhibitor to the formation of naphthenate scale in a liquid hydrocarbon system including:
taking a sample of scale from in situ in the liquid hydrocarbon system;
solubilising a naphthenate solids component of the scale in an organic solvent to form a naphthenate rich organic solvent;
contacting the naphthenate rich organic solvent with an inhibitor and a buffered aqueous solution;
observing the extent of formation of naphthenate solids, if any, the extent of formation of naphthenate solids being indicative of the effectiveness of the inhibitor; and
repeating the steps, if necessary, until a suitable inhibitor is identified.

11. The method of claim 10, wherein the pH of the buffered aqueous solution remains essentially unchanged after contact with the naphthenate rich organic solvent and the inhibitor.

12. The method of claim 10, wherein naphthenic acid is extracted from the scale with an acid and the extracted naphthenic acid is dissolved in the organic solvent to form the naphthenate rich organic solvent.

13. The method of claim 12, including the step of washing the scale with one or more organic solvents prior to acid extraction.

14. The method of claim 10, wherein the concentration of naphthenic acid in the naphthenate rich organic solvent is up to about 1%, preferably between about 0.5% and 1%.

15. The method of claim 10, wherein the step of contacting the naphthenate rich organic solvent with the inhibitor and buffered aqueous solution includes manually shaking the naphthenate rich organic solvent and the inhibitor and buffered aqueous solution, preferably for about 100 shakes.

16. The method of claim 10, wherein the pH of the buffered aqueous solution is between about 7.0 to 8.2 prior to contact with the naphthenate rich organic solvent and the inhibitor.

17. The method of claim 10, including the step of heating the naphthenate rich organic solvent and the inhibitor and buffered aqueous solution at a temperature between about 50° C. and 80° C., preferably for a period of up to 30 minutes.

18. The method of claim 10, including conducting a blank reference test by contacting a sample of the naphthenate rich organic solvent with the buffered aqueous solution in the absence of the inhibitor and observing the extent of reformation of naphthenate solids prior to contacting a sample of the naphthenate rich organic solvent with an inhibitor.

19. The method of claim 10, including assessing the minimum amount of a suitable inhibitor required to inhibit formation of scale by varying the amount of the scale subjected to solubilisation while maintaining a constant concentration of the inhibitor.

20. A test kit for use in a method for identifying an inhibitor to the formation of naphthenate solids in a liquid hydrocarbon, the test kit including:
an acid and conjugate base for buffering an aqueous solution containing one or more ionic species selected from the group consisting of Na⁺, K⁺, Ca²⁺, Mg²⁺, Ba²⁺, Sr²⁺, Cl⁻, SO₄²⁻ and HCO₃⁻ to a pH of 6.4 to 8.2;
a plurality of inhibitors preselected based on the nature of the liquid hydrocarbon; and
at least one vessel in which a sample of liquid hydrocarbon may be contacted with an inhibitor and a buffered aqueous solution formed from the acid, conjugate base and aqueous solution.

21. The test kit of claim 20, wherein the acid and conjugate base include acetic acid and sodium acetate respectively and the aqueous solution is formation water associated with the liquid hydrocarbon to be tested, or is prepared from a synthetic water that includes ionic species at concentrations representative of the formation water associated with the liquid hydrocarbon to be tested.

22. The test kit of claim 20, wherein the inhibitors include at least one linear or cyclic alkoxylated amine, preferably an alkoxylated fatty amine with a carbon chain length from C₁₀-C₂₄, alkyldiamine ethoxylates, tallowalkylamine ethoxylate propoxylates and/or quaternary amines of the type: 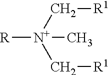
wherein R₁ is (CH₂CH₂O)ₙH and R is a saturated or unsaturated alkyl chain with carbon numbers varying from C₁₀-C₁₆ and having an average number of ethoxylate units of from 10 to 20.

23. The test kit of claim 20, wherein the inhibitors include at least one fatty amine with a carbon chain length between C₁₂-C₂₄.

24. The test kit of claim 20, wherein the method is for identifying an inhibitor to the formation of calcium naphthenate scale in a liquid hydrocarbon system, the test kit including:
an acid;
an organic solvent;
at least a first vessel for solubilising a naphthenate component of the scale into the organic solvent to provide a naphthenate rich organic solvent; and
at least a second vessel in which the naphthenate rich organic solvent may be contacted with an inhibitor and the buffered aqueous solution.

25. The test kit of claim 24, wherein the acid is selected from the group consisting of an organic acid, preferably acetic acid, and an inorganic acid, preferably hydrochloric acid.

26. The test kit of claim 24, wherein the organic solvent is selected from the group consisting of mesitylene, xylene, toluene, heptane and hexane.

27. The test kit of claim 20, wherein in the method for identifying the inhibitor, the pH of the buffered aqueous solution remains essentially unchanged after contact with the sample of the inhibitor and the liquid hydrocarbon or naphthenate rich organic solvent.
